(19) United States
(12) Patent Application Publication
(10) Pub. No.: US 2005/0049220 A1
(43) Pub. Date: Mar. 3, 2005

(54) Dosing regimen for Flaviviridae therapy

(76) Inventor: Lieven J. Stuyver, Herzele (BE)

Correspondence Address: KING & SPALDING LLP, 191 PEACHTREE STREET, N.E., ATLANTA, GA, 30303-1763, US

(21) Appl. No.: 10/921,052

(22) Filed: Aug. 18, 2004

Related U.S. Application Data
(60) Provisional application No. 60/496,202, filed on Aug. 18, 2003.

(51) Int. Cl. A61K 31/7072; A61K 31/66; A61K 31/513; A61K 31/525; A61K 31/522

(52) U.S. Cl. 514/49; 514/251; 514/263.3; 514/269; 514/283; 514/114; 514/449

(57) ABSTRACT

An anti-hepatitis C agent which is an anti-metabolite to the host and cannot be administered on a daily or chronic basis as is usual in anti-viral therapy (referred to below as an “anti-HCV anti-metabolite”), can be administered using a traditional anti-cancer dosing regimen (for example via intravenous or parenteral injection), over a period of one, two, three, four, five, six, or seven days followed by cessation of therapy until rebound of the viral load is noted. This dosing regimen runs counter to conventional antiviral experience, wherein effective agents are usually administered over at least fourteen days of sustained therapy, and typically on an indefinite daily basis.

### CROSS REFERENCE TO RELATED APPLICATIONS

This application claims priority to U.S. Provisional Application No. 60/496,202, filed on Aug. 18, 2003.

### FIELD OF THE INVENTION

The present invention is a dosing regimen for the treatment of a Flaviviridae, such as flavivirus, pestivirus, and notably hepatitis C virus (HCV), with an anti-metabolite which is effective yet may be too toxic for use in a typical antiviral chronic dosing regimen.

### BACKGROUND OF THE INVENTION

Hepatitis C (HCV) was not characterized until 1989. It was originally referred to as non-A, non-B hepatitis. HCV, in combination with hepatitis B, now accounts for 75% of all cases of liver disease worldwide. (Helbling, B., et al., Interferon And Amantadine In Naive Chronic Hepatitis C: A Doubleblind. Randomized. Placebo-Controlled Trial. Hepatology, 2002. 35(2):447-54). It is estimated that approximately 4 million people in the United States are infected with HCV, and more than 200 million persons are infected worldwide. (Hewitt, S. E., Recommendations for Prevention and Control of Hepatitis C Virus (HCV) Infection and HCV-related Disease. 1998, Centers for Disease Control and Prevention). During the 1980's an average of 230,000 new infections occurred each year. After 1989, the number of newly infected individuals declined by >80% to 36,000 by 1986. Most of these persons are chronically infected and may be unaware of their infection because they remain asymptomatic. Thus, HCV-related liver disease is potentially one of the greatest public health threats to be faced in this century. Liver failure related to HCV infection is the leading cause of liver transplants in the United States. The hepatitis C virus (HCV) is the leading cause of chronic liver disease worldwide. (Boyer, N. et al. J. Hepatol. 32:98-112, 2000). Chronic liver disease is the 10th leading cause of death among adults in the United States and accounts for 25,000 deaths annually. Population-based studies estimate that 40% of chronic liver disease is HCV-related. Since most HCV-infected persons are 30-49 years old, the number of deaths associated with HCV-related chronic liver disease may increase substantially over the next 10-20 years. This is not trivial since current medical cost for treating HCV-related complications are estimated to be >600 million dollars annually.

Since HCV infection is typically mild in its early stages, it is rarely diagnosed until its chronic stages. Because of this, HCV is often referred to as the “silent epidemic”. The typical cycle of HCV from infection to symptomatic liver disease takes approximately 20 years, thus the true impact of this disease on the growing infected population will not be apparent for many years. HCV is spread by contact with the blood of an infected person. Individuals with the highest risk factors for HCV infection include:
users of injectable illegal drugs
recipients of blood transfusions or solid organ transplant recipients prior to 1992
recipients of a blood product for clotting problems before 1987
patients on long-term kidney dialysis
individuals that exhibit evidence of liver disease (e.g., persistently abnormal ALT levels)

HCV causes a slow growing viral infection and is the major cause of cirrhosis and hepatocellular carcinoma (Di Besceglie, A. M. and Bacon, B. R., Scientific American, October .: 80-85, (1999); Boyer, N. et al. J. Hepatol. 32:98-112, 2000). An estimated 170 million persons are infected with HCV worldwide. (Boyer, N. et al. J. Hepatol. 32:98-112, 2000). Cirrhosis caused by chronic hepatitis C infection accounts for 8,000-12,000 deaths per year in the United States, and HCV infection is the leading indication for liver transplantation.

HCV is known to cause at least 80% of posttransfusion hepatitis and a substantial proportion of sporadic acute hepatitis. Preliminary evidence also implicates HCV in many cases of “idiopathic” chronic hepatitis, “cryptogenic” cirrhosis, and probably hepatocellular carcinoma unrelated to other hepatitis viruses, such as Hepatitis B Virus (HBV). A small proportion of healthy persons appear to be chronic HCV carriers, varying with geography and other epidemiological factors. The numbers may substantially exceed those for HBV, though information is still preliminary; how many of these persons have subclinical chronic liver disease is unclear. (The Merck Manual, ch. 69, p. 901, 16th ed., (1992)).

HCV is an enveloped virus containing a positive-sense single-stranded RNA genome of approximately 9.4 kb. The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

As an RNA virus, HCV mutates frequently. (www.epidemic.org/index2.html, The Facts about Hepatitis C. 1998, Dartmouth College). Once an infection occurs, HCV creates different genetic variations of itself within the body of the host. The mutated forms frequently differ from their precursors so the immune system cannot recognize them. Thus, even if the immune system succeeds against one variation, the mutant strains quickly take over and become predominate strains. This explains why >80% of individuals infected with HCV will progress to chronic liver disease. HCV has six major genotypes and more than 50 subtypes. In the United States among patients infected with HCV approximately 70% have genotype 1, 15% have genotype 2, and 10% have genotype 3. (McHutchison, J. G., et al., Interferon Alfa-2b Alone Or In Combination With Ribavirin As Initial Treatment For Chronic Hepatitis C. Hepatitis Interventional Therapy Group. N Engl J Med, 1998. 339(21): 1485-92). Antiviral therapy is recommended for patients with chronic HCV infection who are at risk for progression to cirrhosis. (Herrine, S. K., Approach To The Patient With Chronic Hepatitis C Virus Infection. Ann Intern Med, 2002. 136(10):747-57). These persons include anti-HCV-antibody positive patients with persistently elevated ALT levels, detectable HCV RNA, and a liver biopsy that indicates either portal or bridging fibrosis or at least moderate inflammation or necrosis.

A significant focus of current antiviral research is directed to the development of improved methods of treatment of chronic HCV infections in humans (Di Besceglie, A. M. and Bacon, B. R., Scientific American, October.: 80-85, (1999)).

Therapy for HCV is rapidly changing and combination therapy with interferon and ribavirin, a nucleoside analog, is approved in the United States for treatment naïve patients with chronic HCV infection. (Hewitt, S. E., Recommendations for Prevention and Control of Hepatitis C Virus (HCV) Infection and HCV-related Disease. 1998, Centers for Disease Control and Prevention). Sustained response rates have been achieved in 40-50% of patients treated with ribavirin plus interferon compared to 15-25% with interferon alone. However, combination therapy in patients with genotype 1, the most prevalent HCV genotype in the United States, is not very successful and sustained response rates among these patients are still <30%. Furthermore, treatment-related side effects often lead to reductions in dose or discontinuation of treatment. Side effects frequently associated with interferon plus ribavirin therapy include, flu-like symptoms, irritability, depression, anemia, bone marrow suppression and renal failure. Ribavirin is teratogenic and contraindicated in women of child-bearing potential.

Due to the public health threat posed by chronic HCV infection and the limitations of current treatments, there is a growing need for innovative therapeutic approaches to treat HCV infection.

Treatment of HCV Infection with Interferon

Interferons (IFNs) have been commercially available for the treatment of chronic hepatitis for nearly a decade. IFNs are glycoproteins produced by immune cells in response to viral infection. IFNs inhibit replication of a number of viruses, including HCV, and when used as the sole treatment for hepatitis C infection, IFN can in certain cases suppress serum HCV-RNA to undetectable levels. Additionally, IFN can normalize serum amino transferase levels. Unfortunately, the effect of IFN is temporary and a sustained response occurs in only 8%-9% of patients chronically infected with HCV (Gary L. Davis. Gastroenterology 118:S104-S114, 2000). Most patients, however, have difficulty tolerating interferon treatment, which causes severe flu-like symptoms, weight loss, and lack of energy and stamina.

Interferon alpha-2a and interferon alpha-2b are currently approved as monotherapy for the treatment of HCV. ROFERON®-A (Roche) is the recombinant form of interferon alpha-2a. PEGASYS® (Roche) is the pegylated (i.e. polyethylene glycol modified) form of interferon alpha-2a. INTRON®A (Schering Corporation) is the recombinant form of Interferon alpha-2b, and PEG-INTRON® (Schering Corporation) is the pegylated form of interferon alpha-2b.

Other forms of interferon alpha, as well as interferon beta, gamma, tau and omega are currently in clinical development for the treatment of HCV. For example, INFERGEN (interferon alphacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and interferon gamma, interferon tau, and interferon gamma-1b by InterMune are in development.

Ribavirin

Ribavirin (1-β-D-ribofuranosyl-1-1,2,4-triazole-3-carboxamide) is a synthetic, non-interferon-inducing, broad spectrum antiviral nucleoside analog sold under the trade name, Virazole (The Merck Index, 11th edition, Editor: Budavari, S., Merck & Co., Inc., Rahway, N.J., p1304, 1989). U.S. Pat. No. 3,798,209 and RE29,835 disclose and claim ribavirin. Ribavirin is structurally similar to guanosine, and has in vitro activity against several DNA and RNA viruses including Flaviviridae (Gary L. Davis. Gastroenterology 118:S104-S114, 2000).

Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Ribavirin is not approved fro monotherapy against HCV. It has been approved in combination with interferon alpha-2a or interferon alpha-2b for the treatment of HCV.

Combination of Interferon and Ribavirin

The current standard of care for chronic hepatitis C is combination therapy with an alpha interferon and ribavirin. The combination of interferon and ribavirin for the treatment of HCV infection has been reported to be effective in the treatment of interferon naïve patients (Battaglia, A. M. et al., Ann. Pharmacother. 34:487-494, 2000), as well as for treatment of patients when histological disease is present (Berenguer, M. et al. Antivir. Ther. 3(Suppl. 3):125-136, 1998). Studies have show that more patients with hepatitis C respond to pegylated interferon-alpha/ribavirin combination therapy than to combination therapy with unpegylated interferon alpha. However, as with monotherapy, significant side effects develop during combination therapy, including hemolysis, flu-like symptoms, anemia, and fatigue. (Gary L. Davis. Gastroenterology 118:S104-S114, 2000).

Combination therapy with PEG-INTRON® (peginterferon alpha-2b) and REBETOL® (Ribavirin, USP) Capsules is available from Schering Corporation. REBETOL® (Schering Corporation) has also been approved in combination with INTRON® A (Interferon alpha-2b, recombinant, Schering Corporation). Roche's PEGASYS® (pegylated interferon alpha-2a) and COPEGUS® (ribavirin) are also approved for the treatment of HCV.

PCT Publication Nos. WO 99/59621, WO 00/37110, WO 01/81359, WO 02/32414 and WO 03/024461 by Schering Corporation disclose the use of pegylated interferon alpha and ribavirin combination therapy for the treatment of HCV. PCT Publication Nos. WO 99/15194, WO 99/64016, and WO 00/24355 by Hoffmann-La Roche Inc also disclose the use of pegylated interferon alpha and ribavirin combination therapy for the treatment of HCV.

Additional Methods to Treat Flaviviridae Infections

The development of new antiviral agents for Flaviviridae infections, especially hepatitis C, is currently underway. Specific inhibitors of HCV-derived enzymes such as protease, helicase, and polymerase inhibitors are being developed. Drugs that inhibit other steps in HCV replication are also in development, for example, drugs that block production of HCV antigens from the RNA (IRES inhibitors), drugs that prevent the normal processing of HCV proteins (inhibitors of glycosylation), drugs that block entry of HCV into cells (by blocking its receptor) and nonspecific cytoprotective agents that block cell injury caused by the virus infection. Further, molecular approaches are also being developed to treat hepatitis C, for example, ribozymes, which are enzymes that break down specific viral RNA molecules, and antisense oligonucleotides, which are small complementary segments of DNA that bind to viral RNA and inhibit viral replication, are under investigation. A number of HCV treatments are reviewed by Bymock et al. in Antiviral Chemistry & Chemotherapy, 11:2; 79-95 (2000) and De Francesco et al. in Antiviral Research, 58: 1-16 (2003).

Various nucleosides have been developed for the treatment of Flaviviridae infections.

Idenix Pharmaceuticals, Ltd. discloses branched nucleosides, and their use in the treatment of HCV and flaviviruses and pestiviruses in U.S. patent Publication Nos. 2003/0050229 A1, 2004/0097461 A1 2004/0101535 A1, 2003/0060400 A1, 2004/0102414 A1, 2004/0097462 A1, and 2004/0063622 A1 which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier. See also U.S. patent Publication Nos. 2004/0006002 and 2004/0006007 as well as WO 03/026589 and WO 03/026675. Idenix Pharmaceuticals, Ltd. also discloses in U.S. patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, and WO 04/003000. Further, Idenix Pharmaceuticals, Ltd. also discloses in WO 04/046331 Flaviviridae mutations caused by biologically active 2′-branched β-D or β-L nucleosides or a pharmaceutically acceptable salt or prodrug thereof.

Biota Inc. discloses various phosphate derivatives of nucleosides, including 1′, 2′, 3′ or 4′-branched β-D or β-L nucleosides, for the treatment of hepatitis C infection in International Patent Publication WO 03/072757.

Emory University and the University of Georgia Research Foundation, Inc. (UGARF) discloses the use of 2′-fluoronucleosides for the treatment of HCV in U.S. Pat. No. 6,348,587. See also U.S. patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. See also U.S. Pat. Nos. 6,340,690 and 6,605,614; U.S. patent Publication Nos. 2002/0099072 and 2003/0225037, as well as International Publication No. WO 01/32153 and WO 00/50424.

BioChem Pharma Inc. (now Shire Biochem, Inc.) also discloses various other 2′-halo, 2′-hydroxy and 2′-alkoxy nucleosides for the treatment of a Flaviviridae infection in U.S. patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

U.S. Pat. No. 6,660,721; U.S. patent Publication Ser. Nos. 2003/083307 A1, 2003/008841 A1, and 2004/0110718; as well as International Patent Publication Nos. WO 02/18404; WO 02/100415, WO 02/094289, and WO 04/043159; filed by F. Hoffmann-La Roche AG, discloses various nucleoside analogs for the treatment of HCV RNA replication.

Pharmasset Limited discloses various nucleosides and anti-metabolites for the treatment of a variety of viruses, including Flaviviridae, and in particular HCV, in U.S. patent Publication Ser. Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose in U.S. patent Publication No. 2002/0147160, 2004/0072788, 2004/0067901, and 2004/0110717; as well as the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/000858, WO 2004/003138, WO 2004/007512, and WO 2004/009020.

U.S. patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Genelabs Technologies disclose in U.S. patent Publication No. 2004/0063658 as well as International Patent Publication Nos. WO 03/093290 and WO 04/028481 various base modified derivatives of nucleosides, including 1′, 2′, 3′ or 4′-branched β-D or β-L nucleosides, for the treatment of hepatitis C infection.

Eldrup et al. (Oral Session V, Hepatitis C Virus, Flaviviridae; 16^{th} International Conference on Antiviral Research (Apr. 27, 2003, Savannah, Ga.) p. A75) described the structure activity relationship of 2′-modified nucleosides for inhibition of HCV.

Bhat et al (Oral Session V, Hepatitis C Virus, Flaviviridae; 16^{th} International Conference on Antiviral Research (Apr. 27, 2003, Savannah, Ga.); p A75) describe the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

Anti-Metabolites

Folate metabolism is a complex pathway of inter-convertible molecules that, in the presence of adequate reducing enzymes and the vitamins B₆ and B₁₂, results in the regeneration of active form of folic acid. Folic acid is made of up the base pteridine that is attached to one molecule of p-aminobenzoic acid (PABA) and glutamic acid. There are three different pathways involved in folate metabolism: purine synthesis, pyrimidine synthesis, and amino acid interconversions. Because this pathway includes multiple steps, folate metabolism can be terminated at multiple sites. Anti-metabolite drugs such as methotrexate (MTX), raltitrexed, lometrexol (DDATHF) and multitargeted antifolate (MTA) exert their chemotherapeutic roles by inhibiting folate metabolism and ultimately preventing DNA and RNA synthesis.

The idea of synthesizing anti-metabolites came from the fact that folic acid seemed to increase the growth of leukemia cells when given to anemic patients. A folate analog that interferes with folate metabolism, could be useful as a cytotoxic agent (Kamen B. Folate and antifolate pharmacology. Seminars in Oncology (1997) 24 (suppl 18): S18-30-S18-39).

In the intestinal cell, dihydrofolate reductase (DHFR) reduces folic acid to dihydrofolic acid (DHF) and eventually tetrahydrofolic acid (THF). The vitamin then circulates in the plasma until it is either taken up by the cell or excreted in the urine. Cellular uptake is not passive, but is facilitated by transport mechanisms. Two of these include the reduced folate carrier (RCF1) and the folate receptor. RFC1 has a relatively low affinity for natural folates, while the folate receptor has a much higher affinity for the vitamin. Following cellular uptake, the vitamin once again becomes polyglutamated by enzyme folylpoly-γ-glutamate synthetase (FPGS). This enzyme functions to:
improve intracellular retention through metabolic trapping
allow binding of THF to folate-dependent enzymes at lower concentrations than monoglutamate form
increase retention of folate cofactors in the mitochondria and thereby permit a methyl group transfer, which allows the folate metabolism pathway to proceed.
   Dosing Regimens

Although anti-metabolites have been developed as anticancer agents, they have traditionally not been considered as antiviral agents for two reasons (1) anti-metabolites as antitumor agents are generally so toxic that they are usually administered only according to a regimen of typically once a week for three to four weeks followed by a “rest week”; and (ii) standard antiviral therapy consists of daily administration of nucleoside analogues for an indefinite period, and perhaps for the life of the patient.

Antiviral therapy typically requires daily dosing over a long period of time to sustain a 1-2 log drop in viral load. It has been generally accepted by virologists that if therapy with antiviral drugs is stopped (or administered on an infrequent periodic basis) and virus has not been eliminated, the viral load will rebound quickly, and no sustained therapeutic effect will be achieved.

Pharmasset teaches in U.S. patent application No. 2003/0225029 that gemcitabine or its salt, ester or prodrug can be administered in an amount between 50-1300 mg/m² of host surface area, once, twice, three, four, five, six or seven times a day, for treatment of HCV.

U.S. patent application No. 2002/0052317 and WO 02/10743 A1 disclose the use of erythropoietin to improve the tolerance to interferon, and which therapy may optionally also include the administration of one of a generic class of nucleoside analogs, including gemcitabine.

In light of the fact that HCV infection has reached epidemic levels worldwide, and has tragic effects on the infected patient, there remains a strong need to provide compounds, compositions and methods for the treatment of Flaviviridae, and in particular HCV infection, in a host, such as a human.

It is therefore an object of the invention to provide a new regimen which includes a method and composition, for the treatment of Flaviviridae infection, and notably hepatitis C, in an infected host cl SUMMARY OF THE INVENTION

An anti-hepatitis C agent which is an anti-metabolite to the host and cannot be administered on a daily or chronic basis as is usual in anti-viral therapy (referred to below as an “anti-HCV anti-metabolite” or interchangeably “anti-Flaviviridae anti-metabolite”), can be administered using a traditional anti-cancer dosing regimen (for example via intravenous or parenteral injection), over a period of one, two, three, four, five, six, or seven days followed by cessation of therapy until rebound of the viral load is noted. This dosing regimen runs counter to conventional antiviral experience, wherein effective agents are usually administered over at least fourteen days of sustained therapy, and typically on an indefinite daily basis.

The anti-HCV anti-metabolite of the present application can be a chemotherapeutic agent, such as an anti-cancer agent, that is very effective in reducing the viral load of a Flaviviridae, such as HCV, yet too cytotoxic for daily administration for extended periods of time as is traditionally required for viral treatment, or the pharmaceutically acceptable salts or prodrugs, or derivatives thereof.

In one embodiment, the anti HCV anti-metabolite has an EC₅₀ of less than or equal to 5 μM, 4 μM, 3 μM, 2 μM, 1 μM, 0.5 μM, 0.25 μM, or 0.10 μM; and an IC₅₀ of less than or equal to 85 μM, 75 μM, 65 μM, 60 μM, 50 μL, 25 μM, or 10 μM; such that any individual combination of these EC₅₀ and IC₅₀ are possible.

In one particular embodiment of the present invention, the anti-HCV anti-metabolite of the present invention inhibits the de novo biosynthesis of UTP and/or CTP. Non-limiting examples of such anti-metabolites include inosine monophosphate dehydrogenase (IMPDH, E.C.1.1.1.205) inhibitors, aspartate transcarbamoylase (ATC, E.C.2.1.3.2) inhibitors, orotidine 5′-monophosphate decarboxylase (OMPDC, E.C.4.1.1.23) inhibitors, and CTP synthase (CTPS, E.C.6.3.4.2) inhibitors. In a particular embodiment, the anti-metabolites are aspartate transcarbamoylase (ATC, E.C.2.1.3.2) inhibitors, orotidine 5′-monophosphate decarboxylase (OMPDC, E.C.4.1.1.23) inhibitors, or CTP synthase (CTPS, E.C.6.3.4.2) inhibitors.

In a specific embodiment of the present invention, the anti-HCV anti-metabolite is selected from the group consisting of cyclopentylcytosine (CP-C), cyclopentenylcytosine (CPE-C); pyrazofurin (PZF; NSC-143095); and N-(Phosphonoacetyl)-L-aspartate (PALA; NSC-224131).

In an alternative embodiment, the anti-HCV anti-metabolite or its salt, prodrug or derivative is administered according to the regimen described herein in combination or alternation with one or more other anti-Flaviviridae active agent(s). The other active agents (as described in more detail below) are administered in a manner that maximizes their effectiveness in combination with this regimen.

Therefore, the invention provides a method and composition for the treatment of a Flaviviridae infection, and in particular, a hepatitis C viral infection, that includes administering certain anti-HCV anti-metabolites of the present invention (or its salt, prodrug or derivative, as described herein), and particularly anti-metabolites traditionally used as anti-cancer agents, in a dosage range of approximately 50 mg/m² to about 1300 mg/m² per day for one, two or three days, followed by cessation of therapy. Viral load is then optionally monitored over time to evaluate viral rebound. Therapy is not resumed unless a significant viral load is again observed, and then therapy for 1, 2 or 3 days is repeated. This therapy can be continued indefinitely to monitor the and maintain the health of the patient.

Flaviviridae viruses that can be treated include all members of the Hepacivirus genus (HCV), Pestivirus genus (BVDV, CSFV, BDV), and the Flavivirus genus (Dengue virus, Japanese encephalitis virus group (including West Nile Virus), and Yellow Fever virus).

Most studies indicate that HCV genotypes 1a and 1b are more resistant to treatment with any interferon alpha-based therapy than non-type 1 genotypes. For this reason, some doctors may prescribe longer durations of treatment for patients infected with viral genotypes 1a or 1b. Therefore, in one embodiment, the anti-metabolite of the present invention, and particularly anti-metabolites traditionally used as anti-cancer agents, is administered to a patient infected with HCV1a or 1b in doses effective in reducing viral load. Therefore, in one embodiment of the invention, the anti-metabolite is administered to a host carrying HCV genotype 1a or 1b independently of interferon alpha. In a further embodiment, the anti-metabolite of the present invention is administered to a host carrying HCV genotype 1a or 1b in combination with interferon alpha.

In an alternative embodiment, for more severe Flaviviridae infections, the anti-HCV anti-metabolite of the present invention (or its salt, prodrug or derivative, as described herein) is administered in a dosage range of approximately 50 mg/m² to about 1300 mg/m² per day for between one and seven days (e.g. 1, 2, 3, 4, 5, 6, or 7 days) followed by cessation of therapy. Viral load is optionally monitored over time, and after cessation, viral rebound is monitored. Therapy is not resumed unless a significant viral load is again observed, and then therapy for 1-7 days (e.g., independently 1, 2, 3, 4, 5, 6 or 7 days) and more preferred, 1, 2, or 3 days, is repeated. This therapy can be continued indefinitely to monitor the and maintain the health of the patient.

This invention is directed to antiviral therapy with certain anti-HCV anti-metabolites of the present invention or its salt or prodrug that can be achieved using an anti-tumor dosing schedule. In certain embodiments, any approved anti-tumor dosage scheduling for the anti-metabolite can be used to treat a Flaviviridae infection.

In various illustrative and non-limiting embodiments, the daily dosage of the anti-metabolites can range from 100-1500 mg per day, alternatively between 200-1000 mg per day, and more particularly between 300-800 mg per day.

In one illustrative embodiment, on Day 1, the patient is dosed via an intravenous infusion and then asked to remain at the clinic for several hours, up to perhaps 12 hours following administration of the dose of medication. The patient is monitored for safety and tolerance, and blood samples taken to measure HCV-RNA pre-dose, and then at 6 hours and 12 hours post-dose. On Day 2, the patient returns to the clinic for safety assessment and viral load measurements. Optional therapy is continued on days 2, 3, 4, 5, 6 and 7. Therapy is then ceased, and the patient is asked to return to the clinic periodically follow up safety and viral load testing.

It is preferred that the anti-HCV anti-metabolite be administered in the form of an intravenous infusion, because it is known that various anti-metabolites rapidly convert to inactive derivatives in the digestive tract. If it is preferred to administer the anti-metabolite orally, then the compound should preferably be administered in the form of a prodrug that protects the active compound from rapid deactivation without causing an adverse effect on activity.

As an example of the invention, a patient exhibiting multifocal HCC, cirrhosis, and ischaemic hepatitis infected with HCV can be administered 1200 mg of the anti-HCV anti-metabolite in 1000 minutes associated with oxaliplatine. If the tolerance is acceptable, the next day the patient can be given a second dosage of approximately 700 mg of the anti-HCV anti-metabolite.

### BRIEF DESCRIPTION OF THE FIGURES

FIG. 1 is an illustration of various nucleosides and anti-HCV anti-metabolites of the present invention.

FIG. 2 is a graphical depiction of the dynamics of cell growth and HCV RNA levels after exposure to control anti-HCV compounds. HCV replicon cells were seeded at approximately 10⁴ cells per well in a 24-well plate. Over a 7-day period, cells were counted daily, and rRNA and HCV RNA were quantified by Q-RT-PCR. A: IFN-α-2a at 100 IU/ml; B: ribavirin at 100 μM. ●: cell proliferation in absence of compound; ◯: cell proliferation in presence of compound; ▾: HCV RNA levels in untreated cells; ∇: HCV RNA levels in the presence of compound. The curves shown are averages of 3 independent experiments.

FIG. 3 is a graphical depiction of the dynamics of the cell growth and HCV RNA levels after exposure to selected anti-metabolites. Experimental method was as described in FIG. 2. A: PALA at 10 μM; B: PZF at 5 μM; C: CP-C at 25 μM; D: CPE-C at 2.5 μM; ●: cell proliferation in absence of compound; ◯: cell proliferation in presence of compound; ▾: HCV RNA levels in untreated cells; ∇: HCV RNA levels in the presence of compound. The curves are averages of 3 independent experiments.

FIG. 4 is a graphical depiction of the log₁₀ changes for replicon RNA levels per cell. The plots were obtained from data collected in FIGS. 2 and 3, in which log₁₀ changes for cell count and replicon RNA levels were subtracted from each other. A. Control antiviral compounds. ●: no drug control; ▾: IFN-α-2a at 100 IU/ml; ▪: ribavirin at 100 μM. B. Selection of the most important anti-metabolites. ●: no drug control; ▾: dFdC at 50 nM; ▪: CP-C at 25 μM; ♦: CPE-C at 2.5 μM; ▴: PALA at 10 μM; ●: PZF at 5 μM.

### DETAILED DESCRIPTION OF THE INVENTION

The term “anti-HCV anti-metabolite agent” or interchangeably “anti-Flaviviridae anti-metabolite” has used herein refers to a compound that (i) has established anti-hepatitis C or Flaviviridae activity (ii) is an anti-metabolite to the host organism; and (iii) is not a compound described in U.S. Ser. No. 10/367,388 (US 2003/0225029), i.e., is not a β-D or β-L nucleoside of the general formula (I): or its pharmaceutically acceptable salt or prodrug thereof (i.e., gemcitabine or an illustrated derivative thereof) wherein: 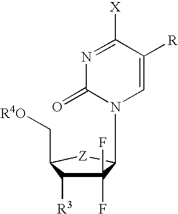
R is H, halogen (F, Cl, Br, I), OH, OR′, SH, SR′, NH 2, NHR′, NR′₂, lower alkyl of C₁-C₆, halogenated (F, Cl, Br, I) lower alkyl of C₁-C₆ such as CF₃ and CH₂CH₂F, lower alkenyl of C₂-C₆ such as CH═CH₂, halogenated (F, Cl, Br, I) lower alkenyl of C₂-C₆ such as CH═CHCl, CH═CHBr and CH═CHl, lower alkynyl of C₂-C₆ such as C≡CH, halogenated (F, Cl, Br, I) lower alkynyl of C₂-C₆, lower alkoxy of C₁-C₆ such as CH₂OH and CH₂CH₂OH, CO₂H, CO₂R′, CONH₂, CONHR′, CONR′₂, CH═CHCO₂H, CH═CHCO₂R′;
X is H, halogen, OH, OR′, OCH₃, SH, SR′, SCH₃, NH2, NHR′, NR_{′2}, CH₃;
each R′ is independently a hydrogen, lower alkyl of C₁-C₆ or lower cycloalkyl of C₁-C₆;
Z is O, S or CH₂;
R⁴ is H, mono-phosphate, di-phosphate, tri-phosphate; a stabilized phosphate prodrug; acyl; alkyl; sulfonate ester; a lipid, a phospholipid; an amino acid; a carbohydrate; a peptide; a cholesterol; or other pharmaceutically acceptable leaving group which when administered in vivo is capable of providing a compound wherein R⁴ is H or phosphate; and
R³ is F or OH.

An anti-hepatitis C agent which is an anti-metabolite to the host and cannot be administered on a daily or chronic basis as is usual in anti-viral therapy (referred to below as an “anti-HCV anti-metabolite”), can be administered using a traditional anti-cancer dosing regimen (for example via intravenous or parenteral injection), over a period of one, two, three, four, five, six, or seven days followed by cessation of therapy until rebound of the viral load is noted. This dosing regimen runs counter to conventional antiviral experience, wherein effective agents are usually administered over at least fourteen days of sustained therapy, and typically on an indefinite daily basis.

The anti-HCV anti-metabolite of the present application can be a chemotherapeutic agent, such as an anti-cancer agent, that is very effective in reducing the viral load of a Flaviviridae, such as HCV, yet too cytotoxic for daily administration for extended periods of time as is traditionally required for viral treatment, or the pharmaceutically acceptable salts or prodrugs, or derivatives thereof.

The invention in particular provides a method and composition for the treatment of a Flaviviridae infection, and in particular, a hepatitis C viral infection, that includes administering an anti-HCV anti-metabolites of the present invention (or its salt, prodrug or derivative, as described herein), and particularly an anti-HCV anti-metabolites traditionally used as anti-cancer agents, in a dosage range of approximately 50 mg/m² to about 1300 mg/m² per day for one, two or three days, followed by cessation of therapy. Viral load is then optionally monitored over time to evaluate viral rebound. Therapy is not resumed unless a significant viral load is again observed, and then therapy for 1, 2 or 3 days is repeated. This therapy can be continued indefinitely to monitor the and maintain the health of the patient.

Flaviviridae viruses that can be treated include all members of the Hepacivirus genus (HCV), Pestivirus genus (BVDV, CSFV, BDV), and the Flavivirus genus (Dengue virus, Japanese encephalitis virus group (including West Nile Virus), and Yellow Fever virus).

Most studies indicate that HCV genotypes 1a and 1b are more resistant to treatment with any interferon alpha-based therapy than non-type 1 genotypes. For this reason, some doctors may prescribe longer durations of treatment for patients infected with viral genotypes 1a or 1b. Therefore, in one embodiment, the anti-metabolite of the present invention, and particularly anti-metabolites traditionally used as anti-cancer agents, is administered to a patient infected with HCV1a or 1b in doses effective in reducing viral load. Therefore, in one embodiment of the invention, the anti-metabolite is administered to a host carrying HCV genotype 1a or 1b independently of interferon alpha. In a further embodiment, the anti-metabolite of the present invention is administered to a host carrying HCV genotype 1a or 1b in combination with interferon alpha.

In an alternative embodiment, for more severe Flaviviridae infections, the anti-metabolite of the present invention (or its salt, prodrug or derivative, as described herein) is administered in a dosage range of approximately 50 mg/m² to about 1300 mg/m² per day for between one and seven days (e.g. 1, 2, 3, 4, 5, 6, or 7 days) followed by cessation of therapy. Viral load is optionally monitored over time, and after cessation, viral rebound is monitored. Therapy is not resumed unless a significant viral load is again observed, and then therapy for 1-7 days (e.g., independently 1, 2, 3, 4, 5, 6 or 7 days) and more preferred, 1, 2, or 3 days, is repeated. This therapy can be continued indefinitely to monitor the and maintain the health of the patient.

This invention is directed to antiviral therapy with anti-HCV anti-metabolites of the present invention or its salt or prodrug that can be achieved using an anti-tumor dosing schedule. In certain embodiments, any approved anti-tumor dosage scheduling for the anti-metabolite can be used to treat a Flaviviridae infection.

Treatment with anti-metabolites results in chemically induced low nucleotide triphosphate pools and cell-cycle arrest in exponentially growing cells. Since steady-state levels of HCV replicon RNA were shown to be dependent on exponential growth of Huh-7 cells, the effect of anti-metabolites for several nucleotide biosynthesis pathways on cell growth and HCV RNA levels was determined. While most anti-metabolites caused a cytostatic effect on cell growth, inhibitors of the de novo pyrimidine ribonucleotide biosynthesis in particular mimicked observations seen in confluent replicon cells, i.e., cytostasis combined with a sharp decrease in replicon copy number per cell. These results suggest that high levels of CTP and UTP are critical parameters for maintaining the steady state level replication of HCV replicon in Huh-7 cells. Therefore, in one embodiment of the invention, the anti-HCV anti-metabolite is an inhibitor of the de novo pyrimidine ribonucleotide biosynthesis.

Anti-Metabolites of the Invention

The anti-HCV anti-metabolite of the present application can be a chemotherapeutic agent, such as an anti-cancer agent, that is very effective in reducing the viral load of a Flaviviridae, such as HCV, yet too cytotoxic for daily administration for extended periods of time as is traditionally required for viral treatment, or the pharmaceutically acceptable salts or prodrugs, or derivatives thereof.

In one embodiment, the anti-HCV anti-metabolite has an EC₅₀ of less than or equal to 5 μM, 4 μM, 3 μM, 2 μM, 1 μM, 0.5 μM, 0.25 μM, or 0.10 μM; and independently an IC₅₀ of less than or equal to 85 μM, 75 μM, 65 μM, 60 μM, 50 μM, 25 μM, or 10 μM.

In a particular embodiment of the present invention, the anti-metabolite is selected from the group consisting of:
Folic Acid Analogs, such as Methotrexate (amethopterin) (acute lymphocytic leukemia, choriocarcinoma, mycosis fungoides, breast, head and neck, lung, osteogenic sarcoma);
Pyrimidine Analogs, such as Fluorouracil (5-fluorouracil; 5-FU), Floxuridine (fluorodeoxyuridine; FUdR) (breast, colon, stomach, pancreas, ovary, head and neck, urinary bladder, premalignant skin lesions) (topical), Cytarabine (cytosine arabinoside) (acute granulocytic and acute lymphocytic leukemias, lymphomatous meningitis), azacitidine (5-azacytidine), raltitrexed, capecitabine (breast, colorectal carcinoma), ibacitabine, fiacitabine (FIAC), zalcitabine, decitabine;
Purine Analogs and Related Inhibitors, such as Mercaptopurine (6-mercaptopurine; 6-MP, Purinethol) (acute lymphocytic, acute granulocytic and chronic granulocytic leukemia), Thioguanine (6-thioguanine, 6-TG) (acute granulocytic, acute lymphocytic and chronic granulocytic leukemia), Pentostatin (2′-deoxycyoformycin) (hairy cell leukemia, mycosis fungoides, chronic lymphocytic leukemia), Azathioprine (Imuran), cladribine (2-CdA) (hairy cell leukemia), fludarabine (B-cell lymphocytic leukemia, CLL);
Vinca Alkaloids, such as Vinblastine (VLB) (Hodgkin's disease, non-Hodgkin's lymphomas, breast, testis), Vincristine (acute lymphocytic leukemia, neuroblastoma, Wilms' tumor, rhabdomyosarcoma, Hodgkin's disease, non-Hodgkin's lymphomas, small-cell lung);
Epipodophylotoxins, such as Etoposide (testis, small-cell lung and other lung, breast, Hodgkin's disease, non-Hodgkin's lymphomas, acute granulocytic leukemia, Kaposi's sarcoma), Teniposide (testis, small-cell lung and other lung, breast, Hodgkin's disease, non-Hodgkin's lymphomas, acute granulocytic leukemia, Kaposi's sarcoma);
Inosine monophosphate dehydrogenase (IMPDH) inhibitors, such as Mizoribine, Tiazofurin, Mycophenolic acid, and C2-MAD;
Ribonucleotide reductase (RNR) inhibitors, such as Guanazole, Hydroxyurea, Tezacytabine, and Deferoxamine;
CTP synthase (CTPS) inhibitors, such as CP-C, CPE-C, 3DU, dFdC;
Orotidine-MP decarboxylase (OMPDC) inhibitors, such as 6-azauridine, 2-thio-6-azauridine, PZF;
Aspartate transcarbamoylase (ATC) inhibitors, such as PALA;
Dihydroorotate dehydrogenase (DHODH) inhibitors, such as Brequinar (NSC-368390), Dichloroallyl lawsone (NSC-126771); and
Thymidylate synthase (TS) inhibitors, such as 2′-deoxy-5-fluorouridine, Methotrexate.

Typically, IMPDH inhibitors showed slight reductions HCV RNA replicon copy number per cell, while CTPS inhibitors were more potent. Thus, intracellular nucleotide pools play an important role in maintaining steady-state levels of HCV RNA copy number. When cells enter anti-metabolites-induced cytostasis, reductions in UTP and CTP levels seem to have a greater impact on reducing HCV RNA turnover than the levels of GTP (or purines).

In one particular embodiment of the present invention, the anti-metabolite inhibits the de novo biosynthesis of UTP and CTP. Non-limiting examples of such anti-metabolites include inosine monophosphate dehydrogenase (IMPDH, E.C.1.1.1.205) inhibitors, aspartate transcarbamoylase (ATC, E.C.2.1.3.2) inhibitors, orotidine 5′-monophosphate decarboxylase (OMPDC, E.C.4.1.1.23) inhibitors, and CTP synthase (CTPS, E.C.6.3.4.2) inhibitors. In a particular embodiment, the anti-metabolites are aspartate transcarbamoylase (ATC, E.C.2.1.3.2) inhibitors, orotidine 5′-monophosphate decarboxylase (OMPDC, E.C.4.1.1.23) inhibitors, or CTP synthase (CTPS, E.C.6.3.4.2) inhibitors.

In a specific embodiment of the present invention, the anti-metabolite is selected from the group consisting of cyclopentylcytosine (CP-C), cyclopentenylcytosine (CPE-C); pyrazofurin (PZF; NSC-143095); and N-(Phosphonoacetyl)-L-aspartate (PALA; NSC-224131).

Definitions

Specific anti-HCV effect was defined as (i) minimal interference with the exponential cell growth, (ii) minimal reduction in cellular host RNA levels, and (iii) reduction of the HCV RNA copy number per cell, as compared to the untreated control in the replicon system.

The term “host,” as used herein, refers to a unicellular or multicellular organism in which the virus can replicate, including cell lines and animals, and preferably a human. Alternatively, the host can be carrying a part of the viral genome, whose replication or function can be altered by the compounds of the present invention. The term host specifically refers to infected cells, cells transfected with all or part of the viral genome and animals, in particular, primates (including chimpanzees) and humans. In most animal applications of the present invention, the host is a human patient. Veterinary applications, in certain indications, however, are clearly anticipated by the present invention (such as bovine viral diarrhea virus in cattle, hog cholera virus in pigs, and border disease virus in sheep).

The term “pharmaceutically acceptable salt or prodrug” is used throughout the specification to describe any pharmaceutically acceptable form (such as an ester, phosphate ester, salt of an ester or a related group) of a compound which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

Pharmaceutical Compositions

Pharmaceutical compositions that include the anti-metabolite as set forth herein or its pharmaceutically acceptable salt or prodrug can be prepared in a therapeutically effective amount for treating a Flaviviridae virus, optionally in combination with a pharmaceutically acceptable additive, carrier or excipient. The therapeutically effective amount may vary with the infection or condition to be treated, its severity, the treatment regimen to be employed, the pharmacokinetics of the agent used, as well as the patient treated.

In one aspect according to the present invention, the compound according to the present invention is formulated preferably in admixture with a pharmaceutically acceptable carrier. In general, it is preferable to administer the pharmaceutical composition in an intravenous form, but formulations may be prepared for administration via oral, parenteral, intramuscular, transdermal, buccal, subcutaneous, suppository or other route. Intravenous and intramuscular formulations are preferably administered in sterile saline. One of ordinary skill in the art may modify the formulation within the teachings of the specification to provide numerous formulations for a particular route. In particular, a modification of a desired compound to render it more soluble in water or other vehicle, for example, may be easily accomplished by routine modification (salt formulation, esterification, etc.).

In certain pharmaceutical dosage forms, for example an oral formulation, the prodrug form of the compound, especially including an acylated (acetylated or other) and ether derivative, phosphate ester or a salt forms of the present compound, is preferred. One of ordinary skill in the art will recognize how to readily modify the present compound to a prodrug form to facilitate delivery of active compound to a targeted site within the host organism or patient. The artisan also will take advantage of favorable pharmacokinetic parameters of the prodrug form, where applicable, in delivering the desired compound to a targeted site within the host organism or patient to maximize the intended effect of the compound in the treatment of Flaviviridae (including HCV) infections.

The amount of compound included within therapeutically active formulations, according to the present invention, is an effective amount for treating a Flaviviridae (including HCV) infection.

Administration of the active compound may range from continuous (intravenous drip) to several oral administrations (for example, Q.I.D., B.I.D., etc.) and may include oral, topical, parenteral, intramuscular, intravenous, subcutaneous, transdermal (which may include a penetration enhancement agent), buccal and suppository administration, among other routes of administration. Enteric-coated oral tablets may also be used to enhance bioavailability and stability of the compounds from an oral route of administration. The most effective dosage form will depend upon the pharmacokinetics of the particular agent chosen, as well as the severity of disease in the patient.

In a first embodiment, the invention provides a method and composition for the treatment of a Flaviviridae infection, and in particular, a hepatitis C viral infection, that includes administering the anti-HCV anti-metabolite or its pharmaceutically acceptable salt or prodrug or derivative in a dosage range of approximately 50 mg/m² to about 1300 mg/m² per day for one, two or three days, followed by cessation of therapy. In an alternative embodiment, for more severe Flaviviridae infections, the anti-HCV anti-metabolite or its pharmaceutically acceptable salt or prodrug or derivative is administered in a dosage range of approximately 50 mg/m² to about 1300 mg/m² per day for between one and seven days (e.g., 1, 2, 3, 4, 5, 6, or 7 days), followed by cessation of therapy.

The daily dosage of the anti-HCV anti-metabolite or another active compound according to the invention can be selected to maximize the therapeutic effect. Examples of nonlimiting dosage ranges are between 100-1500 mg per day, alternatively between 200-1000 mg per day, and more particularly between 300-800 mg per day.

In cases where compounds are sufficiently basic or acidic to form stable nontoxic acid or base salts, administration of the compound as a pharmaceutically acceptable salt may be appropriate. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. In particular, examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including, sulfate, nitrate, bicarbonate, and carbonate salts as well as hydrochloride and hydrobromide salts.

Any of the nucleosides described herein can be administered as a nucleotide prodrug to increase the activity, bioavailability, stability or otherwise alter the properties of the nucleoside. A number of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by refer ence herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stuhmiller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the nucleoside, preferably at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

To prepare the pharmaceutical compositions according to the present invention, a therapeutically effective amount of one or more of the compounds according to the present invention is preferably mixed with a pharmaceutically acceptable carrier according to conventional pharmaceutical compounding techniques to produce a dose. A carrier may take a wide variety of forms depending on the form of preparation desired for administration, e.g., intravenous,or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated for sustained release by standard techniques. The use of these dosage forms may significantly impact the bioavailability of the compounds in the patient.

For parenteral formulations, the carrier will usually comprise sterile water or aqueous sodium chloride solution, though other ingredients, including those that aid dispersion, also may be included. Where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl nucleosides or phosphate ester prodrug forms of the nucleoside compounds according to the present invention.

Combination and/or Alternation Therapies

The active compounds of the present invention can be administered in combination and/or alternation with one or more other anti-Flaviviridae agent(s), such as anti-flavivirus or pestivirus agent(s), or in particular anti-HCV agent(s). In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In preferred embodiments, an anti-HCV (or anti-pestivirus or anti-flavivirus) compound that exhibits an EC₅₀ of 10-15 μM, or preferably less than 1-5 μM, is desirable.

It has been recognized that drug-resistant variants of flaviviruses, pestiviruses or HCV can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Any known anti-HCV agent administered either using conventional anti-viral dosing or by dosing as described herein can be used in combination or alternation with the anti-HCV anti-metabolite dosing strategy described in this specification. Nonlimiting examples include:

(1) Interferon

Interferons (IFNs) are compounds that have been commercially available for the treatment of chronic hepatitis for nearly a decade. IFNs are glycoproteins produced by immune cells in response to viral infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000).

A number of patents disclose HCV treatments using interferon-based therapies. For example, U.S. Pat. No. 5,980,884 to Blatt et al. discloses methods for re-treatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

(2) Ribavirin (Battaglia, A. M. et al., Ann. Pharmacother, 2000,. 34, 487-494); Berenguer, M. et al. Antivir. Ther., 1998, 3 (Suppl. 3), 125-136).

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p1304, 1989); Rebetol (Schering Plough) and Co-Pegasus (Roche). U.S. Pat. No. 3,798,209 and RE29,835 (ICN Pharmaceuticals) disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No 4,211,771 (to ICN Pharmaceuticals) discloses the use of ribavirin as an antiviral agent. Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

Combination of Interferon and Ribavirin

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of Intron®A or PEG-Intron), although Intron A and PEG-Intron are approved for monotherapy (i.e., administration without ribavirin). Hoffman La Roche is selling ribavirin under the name Co-Pegasus in Europe and the United States, also for use in combination with interferon for the treatment of HCV. Other alpha interferon products include Roferon-A (Hoffmann-La Roche), Infergen® (Intermune, formerly Amgen's product), and Wellferon® (Wellcome Foundation) are currently FDA-approved for HCV monotherapy. Interferon products currently in development for HCV include: Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of IFN and ribavirin for the treatment of HCV infection has been reported to be effective in the treatment of IFN naïve patients (for example, Battaglia, A. M. et al., Ann. Pharmacother. 34:487-494, 2000). Combination treatment is effective both before hepatitis develops and when histological disease is present (for example, Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

(3) Protease inhibitors have been developed for the treatment of Flaviviridae infections. Examples, include, but are not limited to the following
Substrate-based NS3 protease inhibitors (see, for example, Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (see, for example, Llinas-Brunet et al, Hepatitis C inhibitor peptide analogues, PCT WO 99/07734);
Non-substrate-based inhibitors such as 2,4,6-trihydroxy-3-nitro-benzamide derivatives (see, for example, Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group;
Phenanthrenequinones possessing activity against protease, for example in a SDS-PAGE and/or autoradiography assay, such as, for example, Sch 68631, isolated from the fermentation culture broth of Streptomyces sp., (see, for example, Chu M. et al., Tetrahedron Letters, 1996, 37, 7229-7232), and Sch 351633, isolated from the fungus Penicillium griseofulvum, which demonstrates activity in a scintillation proximity assay (see, for example, Chu M. et al., Bioorganic and Medicinal Chemistry Letters 9, 1949-1952); and
Selective NS3 inhibitors, for example, based on the macromolecule elgin c, isolated from leech (see, for example, Qasim M. A. et al., Biochemistry, 1997, 36, 1598-1607). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, α-chymotrypsin, chymase and subtilisin.
Several U.S. patents disclose protease inhibitors for the treatment of HCV. Non-limiting examples include, but are not limited to the following. U.S. Pat. No. 6,004,933 to Spruce et al. discloses a class of cysteine protease inhibitors for inhibiting HCV endopeptidase. U.S. Pat. No. 5,990,276 to Zhang et al. discloses synthetic inhibitors of hepatitis C virus NS3 protease. The inhibitor is a subsequence of a substrate of the NS3 protease or a substrate of the NS4A cofactor. The use of restriction enzymes to treat HCV is disclosed in U.S. Pat. No. 5,538,865 to Reyes et al. Peptides as NS3 serine protease inhibitors of HCV are disclosed in WO 02/008251 to Corvas International, Inc, and WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides as NS3 serine protease inhibitors of HCV are disclosed in WO 02/48172 to Schering Corporation. Imidazoleidinones as NS3 serine protease inhibitors of HCV are disclosed in WO 02/08198 to Schering Corporation and WO 02/48157 to Bristol Myers Squibb. WO 98/17679 to Vertex Pharmaceuticals and WO 02/48116 to Bristol Myers Squibb also disclose HCV protease inhibitors.

(4) Thiazolidine derivatives, for example, that show relevant inhibition in a reverse-phase HPLC assay with an NS3/4A fusion protein and NS5A/5B substrate (see, for example, Sudo K. et al., Antiviral Research, 1996, 32, 9-18), especially compound RD-1-6250, possessing a fused cinnamoyl moiety substituted with a long alkyl chain, RD4 6205 and RD4 6193;

(5) Thiazolidines and benzanilides, for example, as identified in Kakiuchi N. et al. J. EBS Letters 421, 217-220; Takeshita N. et al. Analytical Biochemistry, 1997, 247, 242-246;

(6) Helicase inhibitors (see, for example, Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);

(7) Gliotoxin (see, for example, Ferrari R. et al. Journal of Virology, 1999, 73, 1649-1654);

(8) Natural products, such as cerulenin (see, for example, Lohmann V. et al., Virology, 1998, 249, 108-118);

(9) Non-nucleoside polymerase inhibitors, including, for example, compound R803 (see, for example, WO 04/018463 A2 and WO 03/040112 A1, both to Rigel Pharmaceuticals, Inc.); substituted diamine pyrimidines (see, for example, WO 03/063794 A2 to Rigel Pharmaceuticals, Inc.); benzimidazole derivatives (see, for example, Bioorg. Med. Chem. Lett., 2004, 14:119-124 and Bioorg. Med. Chem. Lett., 2004, 14:967-971, both to Boehringer Ingelheim Corporation); N,N-disubstituted phenylalanines (see, for example, J. Biol. Chem., 2003, 278:9495-98 and J. Med. Chem., 2003, 13:1283-85, both to Shire Biochem, Inc.); substituted thiophene-2-carboxylic acids (see, for example, Bioorg. Med. Chem. Lett., 2004, 14:793-796 and Bioorg. Med. Chem. Lett., 2004, 14:797-800, both to Shire Biochem, Inc.); α,γ-diketoacids (see, for example, J. Med. Chem., 2004, 14-17 and WO 00/006529 A1, both to Merck & Co., Inc.); and meconic acid derivatives (see, for example, Bioorg. Med. Chem. Lett., 2004, 3257-3261, WO 02/006246 A1 and WO03/062211 A1, all to IRBM Merck & Co., Inc.);

(10) Antisense phosphorothioate oligodeoxynucleotides (S-ODN) complementary, for example, to sequence stretches in the 5′ non-coding region (NCR) of the virus (see, for example, Alt M. et al., Hepatology, 1995, 22, 707-717), or to nucleotides 326-348 comprising the 3′ end of the NCR and nucleotides 371-388 located in the core coding region of the HCV RNA (see, for example, Alt M. et al., Archives of Virology, 1997, 142, 589-599; Galderisi U. et al., Journal of Cellular Physiology, 1999, 181, 251-257).

(11) Inhibitors of IRES-dependent translation (see, for example, Ikeda N et al., Agent for the prevention and treatment of hepatitis C, Japanese Patent Pub. JP-08268890; Kai Y. et al. Prevention and treatment of viral diseases, Japanese Patent Pub. JP-10101591).

(12) Nuclease-resistant ribozymes (see, for example, Maccjak, D. J. et al., Hepatology 1999, 30, abstract 995; U.S. Pat. No. 6,043,077 to Barber et al., and U.S. Pat. Nos. 5,869,253 and 5,610,054 to Draper et al.).

(13) Various nucleoside analogs that have been developed for the treatment of Flaviviridae infections. Examples include the following.

Idenix Pharmaceuticals, Ltd. discloses branched nucleosides, and their use in the treatment of HCV and flaviviruses and pestivirus in U.S. patent Publication Nos. 2003/0050229 A1, 2004/0097461 A1, 2004/0101535 A1, 2003/0060400 A1, 2004/0102414 A1, 2004/0097462 A1, and 2004/0063622 A1 which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier. See also U.S. patent Publication Nos. 2004/0006002 and 2004/0006007 as well as WO 03/026589 and WO 03/026675. Idenix Pharmaceuticals, Ltd. also discloses in U.S. patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, and WO 04/003000. Further, Idenix Pharmaceuticals, Ltd. also discloses in WO 04/046331 Flaviviridae mutations caused by biologically active 2′-branched β-D or β-L nucleosides or a pharmaceutically acceptable salt or prodrug thereof.

Biota Inc. discloses various phosphate derivatives of nucleosides, including 1′, 2′, 3′ or 4′-branched β-D or β-L nucleosides, for the treatment of hepatitis C infection in International Patent Publication WO 03/072757.

Emory University and the University of Georgia Research Foundation, Inc. (UGARF) discloses the use of 2′-fluoronucleosides for the treatment of HCV in U.S. Pat. No. 6,348,587. See also U.S. patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. See also U.S. Pat. Nos. 6,340,690 and 6,605,614; U.S. patent Publication Nos. 2002/0099072 and 2003/0225037, as well as International Publication No. WO 01/32153 and WO 00/50424.

BioChem Pharma Inc. (now Shire Biochem, Inc.) also discloses various other 2′-halo, 2′-hydroxy and 2′-alkoxy nucleosides for the treatment of a Flaviviridae infection in U.S. patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

U.S. Pat. No. 6,660,721; U.S. patent Publication Nos. 2003/083307 A1, 2003/008841 A1, and 2004/0110718; as well as International Patent Publication Nos. WO 02/18404; WO 02/100415, WO 02/094289, and WO 04/043159; filed by F. Hoffmann-La Roche AG, discloses various nucleoside analogs for the treatment of HCV RNA replication.

Pharmasset Limited discloses various nucleosides and anti-metabolites for the treatment of a variety of viruses, including Flaviviridae, and in particular HCV, in U.S. patent Publication Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose in U.S. patent Publication No. 2002/0147160, 2004/0072788, 2004/0067901, and 2004/0110717; as well as the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/000858, WO 2004/003138, WO 2004/007512, and WO 2004/009020.

U.S. patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Genelabs Technologies disclose in U.S. patent Publication No. 2004/0063658 as well as International Patent Publication Nos. WO 03/093290 and WO 04/028481 various base modified derivatives of nucleosides, including 1′, 2′, 3′ or 4′-branched β-D or β-L nucleosides, for the treatment of hepatitis C infection.

Eldrup et al. (Oral Session V, Hepatitis C Virus, Flaviviridae; 16^{th} International Conference on Antiviral Research (Apr. 27, 2003, Savannah, Ga.) p. A75) described the structure activity relationship of 2′-modified nucleosides for inhibition of HCV.

Bhat et al (Oral Session V, Hepatitis C Virus, Flaviviridae; 16^{th} International Conference on Antiviral Research (Apr. 27, 2003, Savannah, Ga.); p A75) describe the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

(14) Other miscellaneous compounds including 1-amino-alkylcyclohexanes (for example, U.S. Pat. No. 6,034,134 to Gold et al.), alkyl lipids (for example, U.S. Pat. No. 5,922,757 to Chojkier et al.), vitamin E and other antioxidants (for example, U.S. Pat. No. 5,922,757 to Chojkier et al.), squalene, amantadine, bile acids (for example, U.S. Pat. No. 5,846,964 to Ozeki et al.), N-(phosphonoacetyl)-L-aspartic acid (for example, U.S. Pat. No. 5,830,905 to Diana et al.), benzenedicarboxamides (for example, U.S. Pat. No. 5,633,388 to Diana et al.), polyadenylic acid derivatives (for example, U.S. Pat. No. 5,496,546 to Wang et al.), 2′,3′-dideoxyinosine (for example, U.S. Pat. No. 5,026,687 to Yarchoan et al.), benzimidazoles (for example, U.S. Pat. No. 5,891,874 to Colacino et al.), plant extracts (for example, U.S. Pat. No. 5,837,257 to Tsai et al., U.S. Pat. No. 5,725,859 to Omer et al., and U.S. Pat. No. 6,056,961), and piperidenes (for example, U.S. Pat. No. 5,830,905 to Diana et al.).

(15) Other compounds currently in clinical development for treatment of hepatitis C virus include, for example: Interleukin-10 by Schering-Plough, IP-501 by Interneuron, Merimebodib VX-497 by Vertex, AMANTADINE® (Symmetrel) by Endo Labs Solvay, HEPTAZYME® by RPI, IDN-6556 by Idun Pharma., XTL-002 by XTL., HCV/MF59 by Chiron, CIVACIR® (Hepatitis C Immune Globulin) by NABI, LEVOVIRIN® by ICN/Ribapharm, VIRAMIDZINE® by ICN/Ribapharm, ZADAXIN® (thymosin alfa-1) by Sci Clone, thymosin plus pegylated interferon by Sci Clone, CEPLENE® (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc., JTK 003 by AKROS Pharma, BILN-2061 by Boehringer Ingelheim, CellCept (mycophenolate mofetil) by Roche, T67, a β-tubulin inhibitor, by Tularik, a therapeutic vaccine directed to E2 by Innogenetics, FK788 by Fujisawa Healthcare, Inc., IdB 1016 (Siliphos, oral silybin-phosphatdylcholine phytosome), RNA replication inhibitors (VP50406) by ViroPharma/Wyeth, therapeutic vaccine by Intercell, therapeutic vaccine by Epimmune/Genencor, IRES inhibitor by Anadys, ANA 245 and ANA 246 by Anadys, immunotherapy (Therapore) by Avant, protease inhibitor by Corvas/SChering, helicase inhibitor by Vertex, fusion inhibitor by Trimeris, T cell therapy by CellExSys, polymerase inhibitor by Biocryst, targeted RNA chemistry by PTC Therapeutics, Dication by Immtech, Int., protease inhibitor by Agouron, protease inhibitor by Chiron/Medivir, antisense therapy by AVI BioPharma, antisense therapy by Hybridon, hemopurifier by Aethlon Medical, therapeutic vaccine by Merix, protease inhibitor by Bristol-Myers Squibb/Axys, Chron-VacC, a therapeutic vaccine, by Tripep, UT 231B by United Therapeutics, protease, helicase and polymerase inhibitors by Genelabs Technologies, IRES inhibitors by Immusol, R803 by Rigel Pharmaceuticals, INFERGEN® (interferon alphacon-1) by InterMune, OMNIFERON® (natural interferon) by Viragen, ALBUFERON® by Human Genome Sciences, REBIF® (interferon beta-1a) by Ares-Serono, Omega Interferon by BioMedicine, Oral Interferon Alpha by Amarillo Biosciences, interferon gamma, interferon tau, and Interferon gamma-1b by InterMune.

Most studies indicate that HCV genotypes 1a and 1b are more resistant to treatment with any interferon alpha-based therapy than non-type 1 genotypes. For this reason, some doctors may prescribe longer durations of treatment for patients infected with viral genotypes 1a or 1b. Therefore, in one embodiment, the anti-metabolite is administered to a patient infected with HCV1a or 1b in doses effective in reducing viral load. Therefore, in one embodiment of the invention, the anti-metabolite is administered to a host carrying HCV genotype 1a or 1b independently of interferon alpha. In a further embodiment, anti-metabolite is administered to a host carrying HCV genotype 1a or 1b in combination with interferon alpha.

In addition, the compounds according to the present invention can be administered in combination or alternation with one or more antiviral, anti-HIV, anti-HBV, anti-herpetic agent, interferon, anti-cancer and/or antibacterial agents. The preferred compounds include interferon, and in particular interferon alpha, and ribavirin. Certain compounds according to the present invention may be effective for enhancing the biological activity of certain agents according to the present invention by reducing the metabolism, catabolism or inactivation of other compounds and as such, are co-administered for this intended effect.

In an additional embodiment, the method for the treatment or prophylaxis of a mammal having a virus-associated disorder which comprises administering to the mammal a pharmaceutically effective amount of the anti-HCV anti-metabolite, or its pharmaceutically acceptable salt or prodrug thereof, optionally in a combination or alternation with one or more other anti-virally effective agent(s), optionally in a pharmaceutically acceptable carrier or diluent, as disclosed herein, is provided. In a preferred embodiment, the mammal is a human.

In particular, the invention includes methods for treating or preventing and uses for the treatment or prophylaxis of a Flaviviridae infection, including all members of the Hepacivirus genus (HCV), Pestivirus genus (BVDV, CSFV, BDV), or Flavivirus genus (Dengue virus, Japanese encephalitis virus group (including West Nile Virus), and Yellow Fever virus).

This invention is further illustrated in the following sections. The Examples contained therein are set forth to aid in an understanding of the invention. This section is not intended to, and should not be interpreted to, limit in any way the invention set forth in the claims that follow thereafter.

### EXAMPLES

The following working examples provide a further understanding of the method of the present invention. These examples are of illustrative purposes, and are not meant to limit the scope of the invention. Equivalent, similar or suitable solvents, reagents or reaction conditions may be substituted for those particular solvents, reagents or reaction conditions described without departing from the general scope of the method.

### Example 1

### HCV Replicon System

Despite the availability of infectious cDNA clones of the hepatitis C virus (HCV), efficient in vitro replication has not been observed (Bartenschlager, R. and V. Lohmann “Replication of hepatitis C virus” J Gen Virol. 2000, 81, 1631-1648). After transfection of subgenomic HCV RNA replicons that also express the neomycin phosphotranferase gene selection marker, HCV replication has been reported in the human hepatoma cell line Huh-7 (Bartenschlager, R. and V. Lohmann “Novel cell culture systems for the hepatitis C virus” Antiviral Res. 2001, 52, 1-17; Lohmann, V., F. Korner, J. Koch, U. Herian, L. Theilmann, and R. Bartenschlager “Replication of subgenomic hepatitis C virus RNAs in a hepatoma cell line” Science 1999, 285, 110-113). Such HCV replicon-harboring cell lines can be cultivated for more than a year without signs of cytopathogenicity (Pietschmann, T., V. Lohmann, G. Rutter, K. Kurpanek, and R. Bartenschlager “Characterization of cell lines carrying self-replicating hepatitis C virus RNAs” J Virol. 2001, 75, 1252-1264). High levels of HCV RNAs can be maintained in cells passaged under continuous selection with G418. In addition, high-level replication was reflected in the observed adaptations of the HCV replicon to the host cell (Lohmann, V., F. Korner, A. Dobierzewska, and R. Bartenschlager “Mutations in hepatitis C virus RNAs conferring cell culture adaptation” J Virol. 2001, 75, 1437-1449).

Huh7 cells harboring the HCV replicon can be cultivated in DMEM media (high glucose, no pyruvate) containing 10% fetal bovine serum, 1× non-essential Amino Acids, Pen-Strep-Glu (100 units/liter, 100 microgram/liter, and 2.92 mg/liter, respectively) and 500 to 1000 microgram/milliliter G418. Antiviral screening assays can be done in the same media without G418 as follows: in order to keep cells in logarithmic growth phase, seed cells in a 96-well plate at low density, for example 1000 cells per well. Add the test compound immediate after seeding the cells and incubate for a period of 3 to 7 days at 37° C. in an incubator. Media is then removed, and the cells are prepared for total nucleic acid extraction (including replicon RNA and host RNA). Replicon RNA can then be amplified in a Q-RT-PCR protocol, and quantified accordingly. The observed differences in quantification of replicon RNA is one way to express the antiviral potency of the test compound. A typical experiment demonstrates that in the negative control and in the non-active compounds-settings a comparable amount of replicon is produced. This can be concluded because the measured threshold-cycle for HCV RT-PCR in both setting is close to each other. In such experiments, one way to express the antiviral effectiveness of a compound is to subtract the threshold RT-PCR cycle of the test compound with the average threshold RT-PCR cycle of the negative control. This value is called DeltaCt (ΔCt or DCt). A ΔCt of 3.3 equals a 1-log reduction (equals EC₉₀) in replicon production. Compounds that result in a reduction of HCV replicon RNA levels of greater than 2 ΔCt values (75% reduction of replicon RNA) are candidate compounds for antiviral therapy. As a positive control, recombinant interferon alfa-2a (Roferon-A, Hoffmann-Roche, New Jersey, USA) is taken alongside as positive control.

However, this HCV ΔCt value does not include any specificity parameter for the replicon encoded viral RNA-dependent RNA polymerase. In a typical setting, a compound might reduce both the host RNA polymerase activity and the replicon-encoded polymerase activity. Therefore, quantification of rRNA (or any other host RNA polymerase I product) or beta-actin mRNA (or any other host RNA polymerase II) and comparison with RNA levels of the no-drug control is a relative measurement of the effect of the test compound on host RNA polymerases.

With the availability of both the HCV ΔCt data and the rRNA ΔCt, a specificity parameter can be introduced. This parameter is obtained by subtracting both ΔCt values from each other. This results in Delta-DeltaCT values (ΔΔCt or DDCt); a value above 0 means that there is more inhibitory effect on the replicon encoded polymerase, a ΔCt value below 0 means that the host rRNA levels are more affected than the replicon levels. As a general rule, ΔCt values above 2 are considered as significantly different from the no-drug treatment control, and hence, exhibits appreciable antiviral activity. However, compounds with a ΔCt value of less than 2, but showing limited molecular cytotoxicty data (rRNA ΔCT between 0 and 2) are also possible active compounds.

In another typical setting, a compound might reduce the host RNA polymerase activity, but not the host DNA polymerase activity. Therefore, quantification of rDNA or beta-actin DNA (or any other host DNA fragment) and comparison with DNA levels of the no-drug control is a relative measurement of the inhibitory effect of the test compound on cellular DNA polymerases

With the availability of both the HCV ΔCt data and the rDNA ΔCt, a specificity parameter can be introduced. This parameter is obtained by subtracting both ΔCt values from each other. This results in ΔΔCt values; a value above 0 means that there is more inhibitory effect on the replicon encoded polymerase, a ΔΔCt value below 0 means that the host rDNA levels are more affected than the replicon levels. As a general rule, ΔΔCt values above 2 are considered as significantly different from the no-drug treatment control, and hence, is an interested compound for further evaluation. However, compounds with a ΔΔCt value of less than 2, but with limited molecular cytotoxicty (rDNA ΔCt between 0 and 2) may be desired.

Compounds that result in the specific reduction of HCV replicon RNA levels, but with limited reductions in cellular RNA and/or DNA levels are candidate compounds for antiviral therapy. Anti-metabolites were evaluated for their specific capacity of reducing Flaviviridae RNA (including HCV), and potent compounds were detected.

### Example 2

### Specificity of the Antiviral Effect in the HCV Replicon System

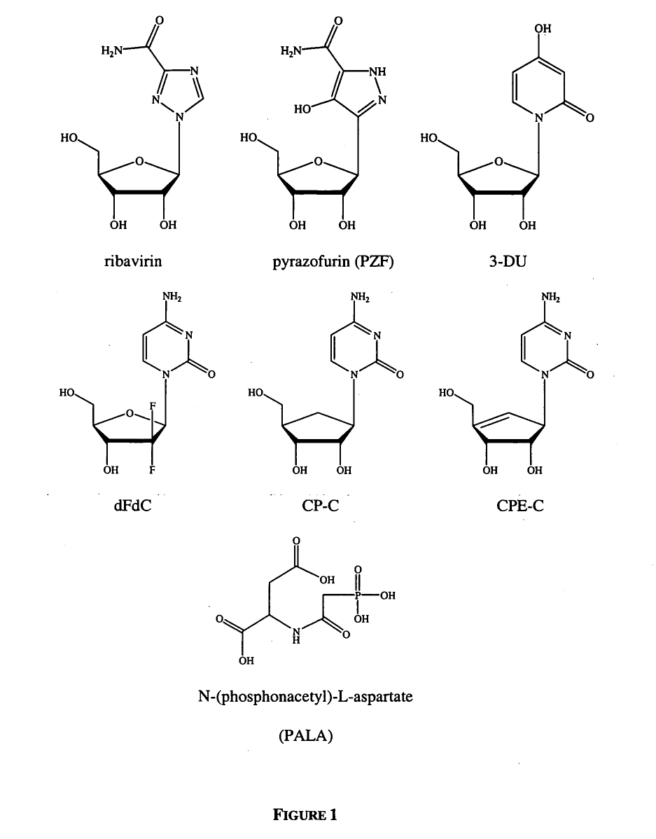

Currently, IFN-α and ribavirin (FIG. 1) are the only drugs approved for treatment of HCV infection. Apart from these two agents, several other compounds have been reported to exert specific antiviral activity against HCV (see for example WO 02/057425 A2 to Merck &Co, Inc. and Isis Pharmaceuticals Inc.; Carroll, S. S. et al. “Inhibition of hepatitis C virus RNA replication by 2′-modified nucleoside analogs” J Biol Chem. 2003, 278, 11979-11984; WO 01/190121 to Idenix Pharmaceuticals; Walker, M. P. and Z. Hong “HCV RNA-dependent RNA polymerase as a target for antiviral development” Curr Opin Pharmacol. 2002, 2, 534-540).

In order to demonstrate the ability of the system to respond to specific inhibition, a series of control experiments were performed. IFN-α-2a and ribavirin were tested over a range of concentrations for their ability to reduce the HCV RNA levels in exponentially growing replicon cells after 4 days of exposure as described (Stuyver, L. et al. “A ribonucleoside analogue that blocks the replication of bovine viral diarrhea and hepatitis C viruses in culture” Antimicrob. Agents Chemother. 2003, 47, 244-254). At 100 IU/mL, IFN-α-2a had a minimal effect on the rRNA levels, and after correcting for this toxicity, a specific antiviral effect of 1.36±0.37 log₁₀ reduction in HCV RNA was observed (Table 1). As previously reported by Stuyver et al., IFN-α-2a showed a corrected EC₉₀ value of 4.5 IU/mL after 96 hr of incubation. Similar experiments and calculations were performed for ribavirin (EC₉₀ value ˜100 μM (Table 1).

However, the EC₉₀ value determined on day 4 is a single static efficacy measurement that does not account for the influence of cell growth dynamics on HCV RNA replication, i.e., the compound related changes in the obligate requirement for logarithmic cell growth. Therefore, experiments were conducted to monitor HCV RNA levels and the cell growth dynamics over a 7-day period.

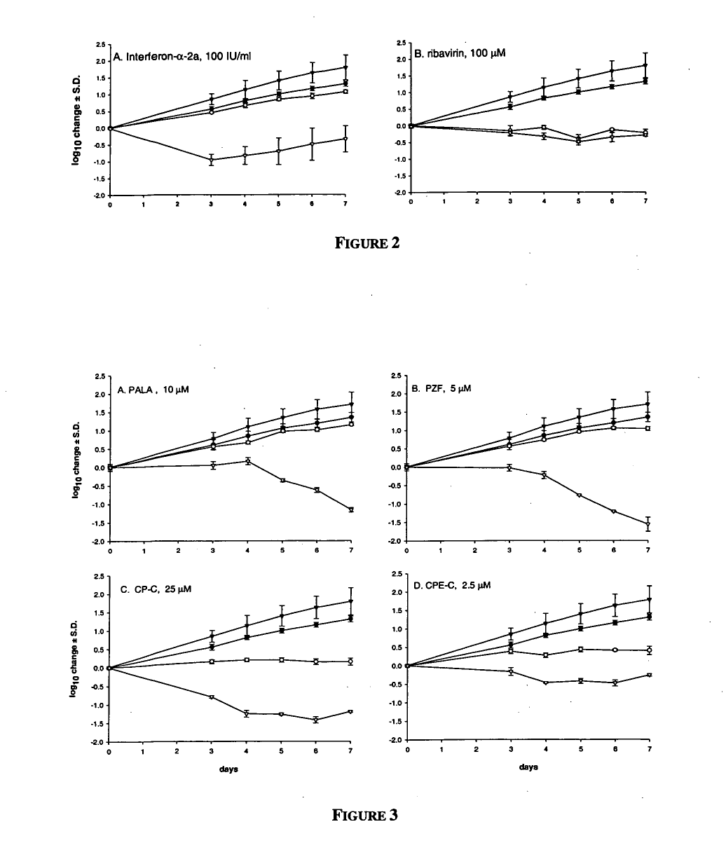

A total of 10⁴ cells per well were seeded in a 24-well plate, and at the end of the incubation step, cells were counted using the trypan-blue exclusion method, and replicon RNA quantified as previously described by Stuyver et al. IFN-α-2a treated cells grew significantly slower (day 7: 1.07±0.06 log₁₀ increase from day 0) than the untreated cell controls (1.31±0.08 log₁₀ increase from day 0; p=0.003) (FIG. 2A). In addition, a significant drop in HCV RNA levels was observed over the 7-day period (control: 1.79±0.4; IFN-α-2a : −0.53±0.4; p=0.0005). The rebound in replicon RNA from day 4 onwards was also noted previously by Cheney et al.(Cheney, I. W., V. C. Lai, W. Zhong, T. Brodhag, S. Dempsey, C. Lim, Z. Hong, J. Y. Lau, and R. C. Tam “Comparative analysis of anti-hepatitis C virus activity and gene expression mediated by alpha, beta, and gamma interferons” J Virol. 2002, 76, 11148-11154).

Ribavirin tested at 100 μM completely inhibited cell proliferation (0.22±0.1 log₁₀ reduction in cell growth at day 7 compared to day 0; or 1.53 log₁₀ reduction compared to the no treatment control on day 7) (FIG. 2B). Although ribavirin reduced HCV RNA by 2.08 log₁₀ on day 7 compared to untreated controls, the ratio of HCV RNA copy number per cell in treatment versus no-treatment controls changed only marginally, suggesting that the antiviral activity is not specific.

Thus, determination of a specific antiviral effect on the HCV RNA replicon depends on at least some, if not a combination of all of the following conditions: (i) no effect on exponential cell growth, (ii) no or limited reduction of cellular host RNA levels, and (iii) significant reduction of HCV RNA copy number per cell, as compared to the untreated controls.

### Example 3

### Antiviral Effect of Anti-Metabolites

One of the most important questions in testing anti-metabolites for anti-replicon (or antiviral) activity is whether induction of the cytostatic and/or cytotoxic condition can be separated from specific antiviral activity, since “dead cells don't make virus”. There are many reports in which specific antiviral activity has been ascribed to anti-metabolites, but to date, no systematic study has been conducted to determine their effects on HCV subgenomic replicon.

The results of our experiments are summarized in Table 1.

TABLE 1
Antiviral and cytotoxicity effects of anti-metabolites on HCV replicon
transfected Huh-7 cells.

| | log₁₀ RNA | | Corrected HCV RNA | | |
|---|---|---|---|---|---|
| | reduction¹ at | | log₁₀ | log₁₀ | Corrected |
| | 100 μM | | reduction¹ | reduction | HCV RNA |
| Compound | HCV | rRNA | at 100 μM | at 10 μM | EC₉₀ (μM) |

Inosine monophosphate dehydrogenase
inhibitors (IMPDH; E.C. 1.1.1.205)

| | | | | | |
|---|---|---|---|---|---|
| Mizoribine | 0.29 ± 0.74 | 0.21 ± 0.50 | 0.08 ± 0.82 | −0.14 ± 0.12 | >100 |
| Tiazofurin | 0.86 ± 0.27 | 0.99 ± 0.35 | −0.13 ± 0.37 | 0.04 ± 0.10 | >100 |
| Mycophenolic acid | 1.15 ± 0.43 | 1.09 ± 0.28 | 0.07 ± 0.47 | 0.22 ± 0.01 | >100 |
| C2-MAD | 1.09 ± 0.21 | 1.00 ± 0.15 | 0.08 ± 0.24 | 0.36 ± 0.21 | >100 |

Ribonucleotide reductase inhibitors
(RNR; E.C. 1.17.4.1; E.C. 1.17.4.2)

| | | | | | |
|---|---|---|---|---|---|
| Guanazole | 0.25 ± 0.11 | 0.07 ± 0.03 | 0.32 ± 0.08 | 0.05 ± 0.08 | >100 |
| Hydroxyurea | 0.17 ± 0.08 | 0.25 ± 0.20 | −0.08 ± 0.16 | 0.06 ± 0.04 | >100 |
| Tezacytabine | 1.59 ± 0.08 | 1.78 ± 0.69 | −0.19 ± 0.49 | 0.63 ± 0.07 | >100 |
| Deferoxamine | 1.00 ± 0.06 | 0.92 ± 0.08 | 0.08 ± 0.03 | 0.17 ± 0.11 | >100 |

CTP synthase inhibitors
(CTPS; E.C. 6.3.4.2.)

| | | | | | |
|---|---|---|---|---|---|
| CP-C | 1.97 ± 0.38 | 0.91 ± 0.13 | 1.06 ± 0.26 | 0.64 ± 0.10 | 25 |
| CPE-C | 2.47 ± 0.33 | 1.21 ± 0.16 | 1.26 ± 0.51 | 1.43 ± 0.01 | 2.5 |
| 3DU | 1.41 ± 0.09 | 0.48 ± 0.11 | 0.94 ± 0.20 | 0.13 ± 0.10 | ˜100 |
| dFdC | 1.87 ± 0.16 | 0.59 ± 0.05 | 1.29 ± 0.11 | 1.32 ± 0.08 | Too toxic |

Orotidine-MP decarboxylase
(OMPDC; E.C. 4.1.1.23)

| | | | | | |
|---|---|---|---|---|---|
| 6-azauridine | 0.25 ± 0.09 | 0.61 ± 0.18 | −0.36 ± 0.16 | 0.12 ± 0.05 | >100 |
| 2-thio-6-azauridine | 0.16 ± 0.04 | −0.02 ± 0.12 | 0.19 ± 0.09 | 0.12 ± 0.10 | >100 |
| PZF | 1.88 ± 0.05 | 0.42 ± 0.03 | 1.46 ± 0.08 | 1.16 ± 0.21 | 3.80 |

Aspartate transcarbamoylase
(ATC; E.C. 2.1.3.2)

| | | | | | |
|---|---|---|---|---|---|
| PALA | 1.77 ± 0.02 | 0.48 ± 0.02 | 1.30 ± 0.05 | 1.18 ± 0.11 | 7.60 |

Dihydroorotate dehydrogenase
(DHODH; E.C. 1.3.3.1)

| | | | | | |
|---|---|---|---|---|---|
| Brequinar (NSC- | −0.05 ± 0.05 | 0.29 ± 0.01 | −0.34 ± 0.04 | −0.17 ± 0.09 | >100 |
| 368390) | | | | | |
| Dichloroallyl | 1.27 ± 0.02 | 2.13 ± 0.15 | −0.86 ± 0.17 | −0.52 ± 0.01 | >100 |
| lawsone (NSC-126771) | | | | | |

Thymidylate synthase
inhibitors (TS; E.C. 2.1.145)

| | | | | | |
|---|---|---|---|---|---|
| 2′-deoxy-5- | 0.76 ± 0.06 | 0.73 ± 0.35 | 0.04 ± 0.25 | 0.23 ± 0.05 | >100 |
| fluorouridine | | | | | |
| Methotrexate | 0.18 ± 0.01 | 0.07 ± 0.10 | 0.11 ± 0.09 | 0.15 ± 0.01 | >100 |

Controls

| | | | | | |
|---|---|---|---|---|---|
| Interferon-α-2a | 1.57 ± 0.26 | 0.21 ± 0.21 | 1.36 ± 0.37 | NA | 4.5 IU/ml |
| Ribavirin | 1.96 ± 0.28 | 0.91 ± 0.12 | 1.05 ± 0.29 | 0.16 ± 0.10 | ˜100 |

¹IFN tested at 100 IU/ml;
dFdC tested initially at 50 μM; a meaningful EC₉₀ value could not be attributed because of extreme toxicity

Several of these anti-metabolites significantly lowered HCV RNA levels. Certain compounds that inhibited enzymes responsible for the de novo biosynthesis of UTP and CTP, such as aspartate transcarbamoylase (ATC, E.C.2.1.3.2), orotidine 5′-monophosphate decarboxylase (OMPDC, E.C.4.1.1.23), and CTP synthase (CTPS, E.C.6.3.4.2) showed modest antiviral activity. EC₉₀ values (96 h incubation) were determined for these compound (FIG. 1) as follows: Cyclopentylcytosine (CP-C)=25 μM; cyclopentenylcytosine (CPE-C)=2.5 μM; 3-deazaurine (3DU) ˜100 μM; pyrazofurin (PZF; NSC-143095)=3.8 μM; N-(Phosphonoacetyl)-L-aspartate (PALA; NSC-224131)=7.6 μM, and 2′-deoxy-2′,2′-difluorocytidine (dFdC, gemcitabine). dFdC was found to be the most potent inhibitor of both replicon RNA and cells. Previous studies have shown that the intracellular metabolites of dFdC exert several antimetabolic activities, including the inhibition of ribonucleotide reductase (RNR) and CTPS (Heinemann, V., L. Schulz, R. D. Issels, and W. Plunkett “Gemcitabine: a modulator of intracellular nucleotide and deoxynucleotide metabolism” Semin Oncol. 1995, 22, 11-18; Plunkett, W., P. Huang, Y. Z. Xu, V. Heinemann, R. Grunewald, and V. Gandhi “Gemcitabine: metabolism, mechanisms of action, and self-potentiation” Semin Oncol. 1995, 22, 3-10).

### Example 4

### Dynamics of the Antiviral Effect of Inhibitors of the De Novo Synthesis of Ribopyrimidines

Exposure of cells to inhibitors of the ATC, OMPDC, and CTPS enzymes can result in reduced levels of UTP and CTP, which subsequently may lead to an arrest in logarithmic cell growth. The dynamics of HCV replicon cells exposed to these inhibitors (at their EC₉₀ value) was monitored over a 7-day period.

When tested at their 96 h EC₉₀ values, PALA and PZF, which are inhibitors of the early de novo pyrimidine biosynthetic steps, reduced cell proliferation only minimally, but significantly reduced HCV RNA levels (FIGS. 3A and B). On the other hand, inhibitors of the CTPS enzyme (last biosynthetic step in the synthesis of CTP) such as CP-C (FIG. 3C), CPE-C (FIG. 3D), and 3-DU (Table 1) caused cytostatic effects on the HCV replicon cell line, but also reduced HCV-replicon RNA levels. Similar levels of cytostatis were also observed with ribavirin (FIG. 2B), although CTPS enzyme inhibitors seemed more specific in reducing HCV RNA levels than IMPDH inhibitors in this cell culture system. DFdC results were meaningless, because a non-cytotoxic concentration (meaning active cell death; range of testing was 50-1,000 nM), could not be found (not shown).

### Example 5

### Reduction of Replicon RNA Copy Number Per Cell

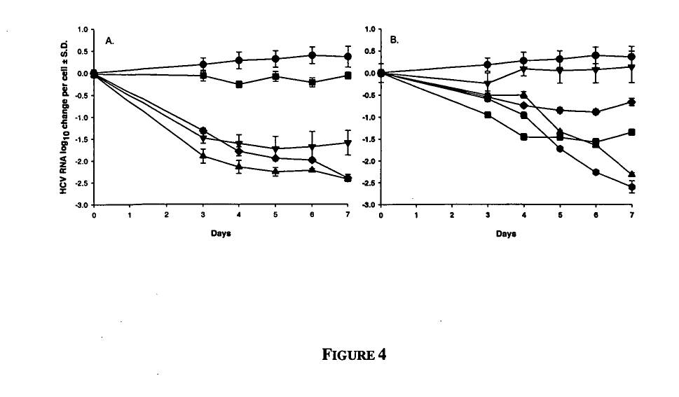

To express the specificity of the antiviral action of the control compounds versus the anti-metabolites, the log₁₀ change in replicon RNA copy number per cell and per day was calculated from the above experiments. IFN-α-2a reduced the copy numbers per cell by approximately 1.6 log₁₀ at day 3, after which a new steady state level was achieved on days 4-7 (FIG. 4A). Ribavirin reduced the replicon RNA copy number per cell only minimally.

The reductions in replicon RNA copy numbers per cell for the anti-metabolites CP-C and CPE-C showed a profile similar to that seen for IFN-α-2a, i.e., a new steady state with a potential rebound at day 7. In contrast, PALA and PZF treatment resulted in a time dependent reduction in replicon RNA levels, with a reduction of 2.96 log₁₀ after 7 days treatment with 5 μM PZF (FIG. 4B).

### Example 6

### Prevention Studies

To study the possibility of preventing the observed antiviral and cytostatic effects, cells were incubated simultaneously with the test compound (at 96 h EC₉₀ value) and the natural ribo- or 2′-deoxy nucleosides (at 50 μM). The antiviral effect of IFN-α-2a could not be prevented by any of the natural nucleosides at this concentration. As expected for the IMPDH inhibitors, 2′-deoxyguanosine and guanosine prevented the effects of ribavirin on cell growth and HCV replicon RNA replication. For dFdC, the observed toxicities and antiviral effects were prevented by 2′-deoxycytidine. In line with expectations for CTPS inhibitors (CPE-C, CP-C, and 3-DU), addition of cytidine to the culture medium compensated for the inhibitory effects. Surprisingly, the antimetabolic effects of CPE-C could be prevented by both 50 μM uridine and cytidine in the media. The effects of PALA and PZF could be prevented by addition of uridine to the culture media. These results indeed indicate that (i) the intracellular levels of uridine and cytidine (most likely at the 5′-TP form) are indeed responsible for the antiviral activity, and for the cytostatic effect, and/or (ii) the activity of the anti-metabolites depends upon steps in the uridine/cytidine pathway.

### Example 7

### Antiviral Activity of Gemcitabine dFdC

Gemcitabine was dissolved in DMSO and added to the culture media of a cellular model system of Huh7 cells harboring self-replicating HCV RNA, at final concentrations ranging from 0.1 to 50 dM. In such experiments, one way to express the antiviral effectiveness of a compound is to subtract the threshold reverse-transcriptase polymerase chain reactions (RT-PCR) cycle of the test compound with the average threshold RT-PCR cycle of the negative control. This value is called DeltaCt (ΔCt or dCt). With the availability of both the HCV ΔCt data and the rRNA ΔCt, a specificity parameter can be introduced. This parameter is obtained by subtracting both ΔCt values from each other. This results in Delta-DeltaCT values (ΔΔCt or ddct). A 4-days incubation resulted in dose-dependant reduction of the replicon HCV RNA (FIG. 2). Since 3.3 Ct values equals 1-log reduction of replicon RNA, an EC₉₀ value was reached at approximately 70 nM. Further analysis of the reduction of cellular DNA levels (ribosomal DNA) or cellular RNA levels (ribosomal RNA) resulted in a dCt that expressed the inhibitory capacity of this compound on host DNA and RNA polymerases. Based on these calculations, In a cellular model system of Huh7 cells harboring self-replicating HCV RNA, gemcitabine significantly reduced HCV RNA levels (EC₅₀=0.040 μM) at a concentration below the IC₅₀ (0.240 μM). Interestingly, the inactive metabolite dFdU (7.0 μM) demonstrated similar activity to dFdC in the HCV replicon system [dCT HCV =6.39, dCt rRNA=1.96, and ddCt: 4.42; (EC₅₀ and IC₅₀ data not available)].

### Example 8

### Antiviral Activity of Gemcitabine After Single Treatment in Human

A male patient exhibiting multifocal HCC, cirrhosis, and ischaemic hepatitis infected with HCV was administered 1200 mg gemcitabine HCl in 1000 minutes associated with oxaliplatine. The tolerance was acceptable, and thus the next day the patient was given a second dosage of approximately 700 mg of gemcitabine. Before the second dosage the baseline viral load was 6.49 log copies/mL. The second perfusion of gemcitabine was stopped after approximately 700 mg because of heart problems. The HCV RNA measurement eight hours after the second dosage was 4.04 log copies/mL, indicating an approximate 2.5 log drop in eight hours.

This invention has been described with reference to its preferred embodiments. Variations and modifications of the invention will be obvious to those skilled in the art from the foregoing detailed description of the invention. It is intended that all of these variations and modifications be included within the scope of this invention.

1. A method for the treatment of a host infected with a hepatitis C virus, comprising administering an “anti-HCV anti-metabolite” over a period of one, two, three, four, five, six, or seven days followed by cessation of therapy until rebound of the viral load is noted.

2. The method of claim 1, wherein the anti-HCV antimetabolite is administered
a) in an amount between 50-1300 mg/m² of host surface area; and
b) in a dosage regimen of daily for one, two, three, four, five, six or seven consecutive days followed by cessation of therapy.

3. The method of claim 1, wherein the anti-HCV anti-metabolite that is selected from the group consisting of methotrexate, 5-fluorouracil, 2′-deoxy-5-fluorouridine, cytosine arabinoside, 5-azacytidine, raltitrexed, capecitabine, ibacitabine, fiacitabine, zalcitabine, decitabine, 6-mercaptopurine, 6-thioguanine, 2′-deoxycyoformycin, azathioprine, cladribine, fludarabine, vinblastine, vincristine, etoposide, teniposide, mizoribine, tiazofurin, mycophenolic acid, C2-MAD, guanazole, hydroxyurea, tezacytabine, deferoxamine, cyclopentylcytosine (CP-C), cyclopentenylcytosine (CPE-C), 3DU, dFdC, 6-azauridine, 2-thio-6-azauridine, pyrazofurin (PZF), N-(Phosphonoacetyl)-L-aspartate (PALA), brequinar, and dichloroallyl lawsone, or its pharmaceutically acceptable salt or prodrug, optionally in a pharmaceutically acceptable carrier or diluent.

4. The method of claim 1, wherein the anti-HCV anti-metabolite is selected from the group consisting of mizoribine, tiazofurin, cycophenolic. acid, C2-MAD, 6-azauridine, 2-thio-6-azauridine, 3DU, dFdC, cyclopentylcytosine (CP-C), cyclopentenyl-cytosine (CPE-C), pyrazofurin (PZF), and N-(Phosphonoacetyl)-L-aspartate (PALA).

5. The method of claim 1, wherein the anti-HCV anti-metabolite is selected from the group consisting of cyclopentylcytosine (CP-C), cyclopentenylcytosine (CPE-C), pyrazofurin (PZF), and N-(Phosphonoacetyl)-L-aspartate (PALA).

6. The method of claim 1, wherein the anti-HCV anti-metabolite is administered in an amount between 200-1000 mg/m² per day.

7. The method of claim 1, wherein the anti-HCV anti-metabolite is administered in an amount between 300-800 mg/m² per day.

8. The method of claim 1, wherein the dosage regimen is once a day for one day.

9. The method of claim 1, wherein the dosage regimen is once a day for two days.

10. The method of claim 1, wherein the dosage regimen is once a day for three days.

11. The method of claim 1, wherein the dosage regimen is once a day for four days.

12. The method of claim 1, wherein the dosage regimen is once a day for five days.

13. The method of claim 1, wherein the dosage regimen is once a day for six days.

14. The method of claim 1, wherein the dosage regimen is once a day for seven days.

15. The method of claim 1, wherein the therapy is ceased for at least two days.

16. The method of claim 1, wherein the therapy is ceased for at least three days.

17. The method of claim 1, wherein the therapy is ceased for at least one week.

18. The method of claim 1, wherein the therapy is ceased for at least two weeks.

19. The method of claim 1, wherein the therapy is ceased for at least three weeks.

20. The method of claim 1, wherein the therapy is ceased for at least one month.

21. The method of claim 1, wherein the host is a human.

22. The method of claim 1, wherein the anti-HCV anti-metabolite is administered orally.

23. The method of claim 1, wherein the anti-HCV anti-metabolite is administered parenterally.

24. The method of claim 1, wherein the anti-HCV anti-metabolite is administered intravenously.

25. A method for the treatment of a host infected with a Flaviviridae, comprising administering an “anti-Flaviviridae anti-metabolite” over a period of one, two, three, four, five, six, or seven days followed by cessation of therapy until rebound of the viral load is noted.

26. The method of claim 25, wherein the anti-Flaviviridae antimetabolite is administered
a) in an amount between 50-1300 mg/m² of host surface area; and
b) in a dosage regimen of daily for one, two, three, four, five, six or seven consecutive days followed by cessation of therapy.

27. The method of claim 25, wherein the anti-Flaviviridae anti-metabolite that is selected from the group consisting of methotrexate, 5-fluorouracil, 2′-deoxy-5-fluorouridine, cytosine arabinoside, 5-azacytidine, raltitrexed, capecitabine, ibacitabine, fiacitabine, zalcitabine, decitabine, 6-mercaptopurine, 6-thioguanine, 2′-deoxycyoformycin, azathioprine, cladribine, fludarabine, vinblastine, vincristine, etoposide, teniposide, mizoribine, tiazofurin, mycophenolic acid, C2-MAD, guanazole, hydroxyurea, tezacytabine, deferoxamine, cyclopentylcytosine (CP-C), cyclopentenylcytosine (CPE-C), 3DU, dFdC, 6-azauridine, 2-thio-6-azauridine, pyrazofurin (PZF), N-(Phosphonoacetyl)-L-aspartate (PALA), brequinar, and dichloroallyl lawsone, or its pharmaceutically acceptable salt or prodrug, optionally in a pharmaceutically acceptable carrier or diluent.

28. The method of claim 25, wherein the anti-Flaviviridae anti-metabolite is selected from the group consisting of mizoribine, tiazofurin, cycophenolic acid, C2-MAD, 6-azauridine, 2-thio-6-azauridine, 3DU, dFdC, cyclopentylcytosine (CP-C), cyclopentenyl-cytosine (CPE-C), pyrazofurin (PZF), and N-(Phosphonoacetyl)-L-aspartate (PALA).

29. The method of claim 25, wherein the anti-Flaviviridae anti-metabolite is selected from the group consisting of cyclopentylcytosine (CP-C), cyclopentenylcytosine (CPE-C), pyrazofurin (PZF), and N-(Phosphonoacetyl)-L-aspartate (PALA).

30. The method of claim 25, wherein the anti-Flaviviridae anti-metabolite is administered in an amount between 200-1000 mg/m² per day.

31. The method of claim 25, wherein the anti-Flaviviridae anti-metabolite is administered in an amount between 300-800 mg/m² per day.

32. The method of claim 25, wherein the dosage regimen is once a day for one day.

33. The method of claim 25, wherein the dosage regimen is once a day for two days.

34. The method of claim 25, wherein the dosage regimen is once a day for three days.

35. The method of claim 25, wherein the dosage regimen is once a day for four days.

36. The method of claim 25, wherein the dosage regimen is once a day for five days.

37. The method of claim 25, wherein the dosage regimen is once a day for six days.

38. The method of claim 25, wherein the dosage regimen is once a day for seven days.

39. The method of claim 25, wherein the therapy is ceased for at least two days.

40. The method of claim 25, wherein the therapy is ceased for at least three days.

41. The method of claim 25, wherein the therapy is ceased for at least one week.

42. The method of claim 25, wherein the therapy is ceased for at least two weeks.

43. The method of claim 25, wherein the therapy is ceased for at least three weeks.

44. The method of claim 25, wherein the therapy is ceased for at least one month.

45. The method of claim 25, wherein the Flaviviridae is hepatitis C virus.

46. The method of claim 25, wherein the Flaviviridae is West Nile Virus.

47. The method of claim 25, wherein the Flaviviridae is Dengue virus.

48. The method of claim 25, wherein the Flaviviridae is Bovine Viral Diarrhea Virus.

49. The method of claim 25, wherein the Flaviviridae is Border Disease Virus.

50. The method of claim 25, wherein the Flaviviridae is Yellow Fever virus.

51. The method of claim 25, wherein the anti-Flaviviridae anti-metabolite is administered orally.

52. The method of claim 25, wherein the anti-Flaviviridae anti-metabolite is administered parenterally.

53. The method of claim 25, wherein the anti-Flaviviridae anti-metabolite is administered intravenously.

54. The method of claim 25, wherein the host is a human.

55. The method of claim 1 or 25, wherein the anti-metabolite is administered in combination and/or alternation with one or more other antivirally effective agents.

56. The method of claim 55, wherein the other antivirally effective agent is selected from the group consisting of an interferon, ribavirin, an interleukin, an NS3 protease inhibitor, a cysteine protease inhibitor, phenanthrenequinone, a thiazolidine derivative, a thiazolidine and a benzanilide, a helicase inhibitor, a polymerase inhibitor, a nucleotide analogue, gliotoxin, cerulenin, an antisense phosphorothioate oligodeoxynucleotide, an inhibitor of IRES-dependent translation, and a ribozyme.

57. The method of claim 55, wherein the additional antivirally effective agent is an interferon.

58. The method of claim 55, wherein the additional antivirally effective agent is selected from the group consisting of pegylated interferon alpha 2a, interferon alphacon-1, natural interferon, albuferon, interferon beta-1a, omega interferon, interferon alpha, interferon gamma, interferon tau, interferon delta and interferon gamma-1b.
